(19) United States
(12) Patent Application Publication
(10) Pub. No.: US 2016/0060587 A1
(43) Pub. Date: Mar. 3, 2016

(54) SYSTEM AND METHOD FOR PRODUCING A COMESTIBLE PROTEIN MEAL AND FUEL FROM A FEEDSTOCK

(71) Applicant: Freddie Hebert, Gueydan, LA (US)

(72) Inventor: Freddie Hebert, Gueydan, LA (US)

(21) Appl. No.: 14/936,724

(22) Filed: Nov. 10, 2015

Related U.S. Application Data
(63) Continuation-in-part of application No. 14/474,334, filed on Sep. 2, 2014.
(60) Provisional application No. 61/878,007, filed on Sep. 15, 2013.

(51) Int. Cl. C12M 1/00; C12M 1/33; C12M 1/02; A23L 1/305; G01N 33/68; C05F 5/00; C10L 1/02; C12P 7/14; C12M 1/34

(52) U.S. Cl. CPC C12M 21/12; C12P 7/14; C12M 43/02; C12M 45/02; C12M 41/18; C12M 41/32; C12M 47/14; C12M 47/10; G01N 33/6803; C05F 5/008; C10L 1/02; A23L 1/3051; G01N 2333/415; C10L 2290/26; C10L 2290/08; C10L 2290/28; C10L 2290/543

(57) ABSTRACT

A method for making comestible protein meal includes: producing an alcohol based fuel (such as ethanol) from a starch-based plant feedstock (such as duckweed); then separating stillage that remains afterwards into solids and a thin stillage; drying the solids to a comestible protein meal (minimum 40% protein); and recycling the thin stillage as plant fertilizer. Another method includes: determining a desired amino acid profile; while producing ethanol from a plant feedstock in a process that comprises a fermentation stage, adding a reagent prior to the fermentation stage to adjust an amount of an amino acid output from the fermentation stage to match an amount of the amino acid in the desired amino acid profile; separating stillage remaining after producing the ethanol into solids and a thin stillage; and drying the solids to a comestible protein meal comprising the amount of the amino acid.

### CROSS REFERENCE TO RELATED APPLICATION

This is a continuation-in-part of U.S. patent application Ser. No. 14/474,334 (filed on Sep. 2, 2014) which itself claims benefit of US Provisional Patent Application Ser. No. 61/878,007 (filed on Sep. 15, 2013). The contents of U.S. patent application Ser. No. 14/474,334 are hereby incorporated in its entirety.

### TECHNICAL FIELD

This invention relates generally to the commercial production of protein meal for human and/or animal consumption from a feedstock such as duckweed whereby a fuel such as ethanol is a byproduct of the production process.

### BACKGROUND

There are a variety of sources from which high quality protein concentrates (generally in the form of a powdered protein meal) for human and animal consumption are produced. In general the livestock feed industry is best able to optimize the production cost per unit volume of protein meal because they deal in very large quantities of protein meal and their livestock-raising customers are more price sensitive while being adaptable to market substitutes. Cattle ranchers, hog farmers, fish farm operators and the like are examples of these livestock-raising customers. Typically these customers purchase their feed from a feed mill that custom mixes different feeds for different animals from raw protein meal and other ingredients they purchase in bulk.

Generally the costliest ingredient is the raw protein meal, which can come from a variety of sources. Soybean is a major one of those sources if not the major source. The soybean meal market is so substantial that soybean meal is traded on commodity exchanges worldwide, with many exchanges separating organic from non-organic protein meal to support the growing market for organic foods. Commodity grade soybean meal is generally 48% protein, which is highly concentrated.

Due to the climate in which soybeans are typically grown and in view of the burgeoning organic foods market, feed mills are often challenged in finding adequate supplies of raw organic protein meal during the winter months. This can result in limiting the size of organic livestock herds and consequently increased prices for organic meats for a given level of demand.

There are also environmental challenges with growing soybeans, particularly water usage and fertilization. Like most agriculture they are water intensive and so crop yield is subject to both drought and excessive rains at the wrong time of the growth cycle, but increasingly natural underground aquifers in major soybean growing areas such as the American Midwest region are being depleted. Some expanding population centers in water-scarce regions such as California are questioning the wisdom of long-established water allocations for agricultural purposes. And like most agriculture soybean farming requires both fertilization and pest management, both of which involve a balancing of cost versus environmental impact.

What is needed in the art is a raw protein meal that can compete with soybean meal in price and volume and an economical way to produce it, preferably while overcoming at least some of the disadvantages in growing soybeans that are noted above.

### SUMMARY

In a first aspect, embodiments of these teachings are manifest in a processing facility comprising: milling equipment, sugaring process equipment, distillation equipment, separation equipment, drying equipment and liquid retention equipment with one or more nitrogen sensors. The milling equipment is arranged to reduce particle size of a feedstock and rupture cell walls of the feedstock. The sugaring process equipment is arranged to cook outputs from the milling equipment so as to convert starch in said outputs to sugar. The fermentation equipment is arranged to ferment outputs of the sugaring process equipment so as to convert sugar in said outputs to protein while converting elemental nitrogen to nitrate and/or nitrite. The distillation equipment is arranged to strip an alcohol based fuel from outputs of the fermentation equipment. The separation equipment is arranged to separate solids and liquids; it separates these from outputs of the distillation equipment other than the stripped fuel, and also from outputs of the fermentation equipment (since not all outputs of the fermentation equipment need pass through the distillation equipment). The drying equipment is arranged to dry solids output from the separation equipment. And the liquid retention equipment is arranged to store liquids output from the separation equipment, and this liquid retention equipment further has one or more nitrogen sensors to measure levels of nitrate and/or nitrite in the stored liquid.

In a second aspect, embodiments of these teachings are manifest in a method for producing protein meal that is comestible by humans and/or farm animals and/or fish. In this aspect the method comprises: producing an alcohol based fuel (such as but not limited to ethanol) from a starch-based plant feedstock (such as but not limited to duckweed); separating stillage that remains after producing the alcohol based fuel into solids and a thin stillage; drying the solids to a comestible protein meal having at least about 40% protein content by weight; and recycling the thin stillage as plant fertilizer.

In a third aspect, embodiments of these teachings are manifest in another method for producing protein meal that is comestible by humans and/or farm animals and/or fish. In this aspect the method comprises: determining a desired amino acid profile; while producing an alcohol based fuel from a starch-based plant feedstock in a process that comprises a fermentation stage, adding at least one reagent prior to the fermentation stage so as to adjust an amount of at least one amino acid output from the fermentation stage to match an amount of the at least one amino acid in the desired amino acid profile; separating stillage, containing the amount of the at least one amino acid, that remains after producing the alcohol based fuel into solids and a thin stillage; and drying the solids to a comestible protein meal comprising the amount of the at least one amino acid.

### BRIEF DESCRIPTION OF THE DRAWINGS

FIG. 1 is a conceptual diagram illustrating some of the major components in a closed loop protein and ethanol production process in accordance with certain embodiments of these teachings.

FIG. 2 is a diagram of grow ponds for an aquatic feedstock as part of the overall system according to certain embodiments of these teachings.

FIG. 3 is a high level process diagram illustrating various steps in the production of ethanol and protein meal from a starch based feedstock in accordance with certain embodiments of these teachings.

FIG. 4 is a logic flow diagram summarizing a method according to certain embodiments of this invention.

### DETAILED DESCRIPTION

FIG. 1 is a conceptual diagram illustrating some of the major components in a closed loop protein and ethanol production process. The process can use any starch-based plant as the feedstock and the amount of starch in the feedstock will generally corresponds to the concentration of protein in the end product protein meal so feedstocks with higher quantities of starch are preferable. Starch based in this case means a minimum of about 35% starch in the feedstock used in the process. The examples herein use the aquatic plant duckweed 100 (Spirodela Polyrhiza) as the feedstock for that reason and because it grows quickly (doubling in size in 2-3 days), it utilizes high levels of nitrogen to grow, and being an aquatic plant it can be harvested very economically as compared to soil-grown plants.

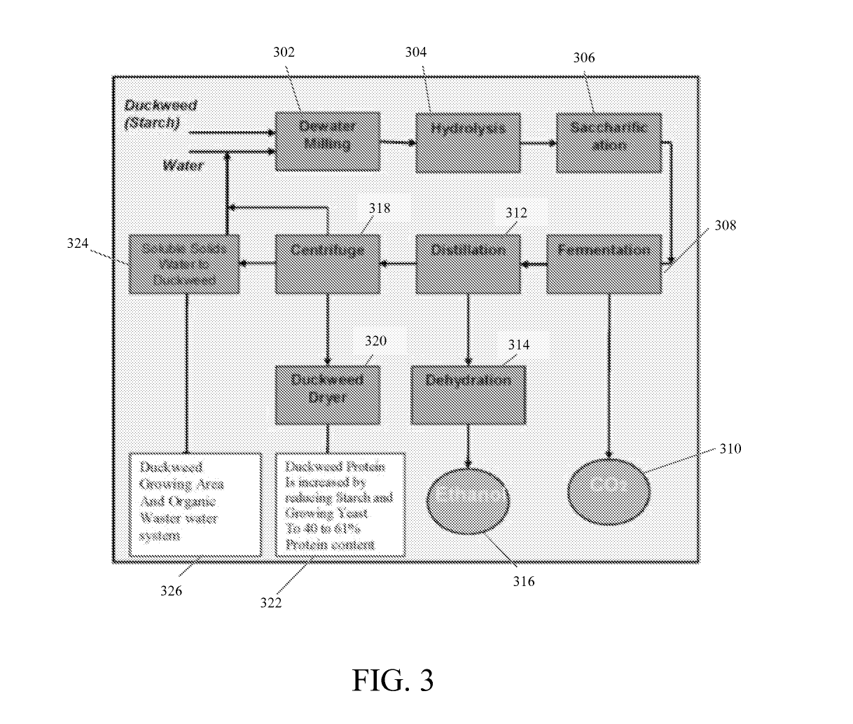

As will be shown in further detail at FIG. 3, producing a fuel in the form of ethanol 102 as well as protein meal 103 from a duckweed feedstock 101 requires energy to run the processing plant which FIG. 1 shows as biomass electricity 104. Particularly the freeze drying portion of the protein meal production is quite energy intensive. While the needed electricity can be obtained from several sources including from the conventional electrical grid, it is preferable to utilize on-site generation of the needed electricity and this can be done via traditional boiler/steam turbine generators or more recently commercialized small scale ‘black-box’ type nuclear powered generators.

A traditional boiler/steam turbine generator can be fueled by the ethanol produced from the process itself or any other commercially available fuel. This description considers a complete commercial operation at which the feedstock 101 is grown on site or nearby the processing plant that produces the fuel 102 and protein meal 103 from the feedstock 101. Due to the amount of land required for such an operation would generally be located in an agricultural area in which case often there will be a steady stream of biomass waste from other agricultural operations, for example rice hulls from rice processors, nut shells from peanut/almond/other nut processors, and the like. For increased economy in operating such a facility FIG. 1 assumes such waste biomass from external sources is burned to heat water in a boiler whose steam is used to run the turbine that generates electricity for the facility, leaving the ethanol 102 or other fuel produced by the process described herein available to be sold at market. For a 600 acre plant with 480 acres used for duckweed grow ponds a 3 MW generator is sufficient to provide all the needed electrical power to the facility. The inventor's projections use a 5 MW generator to provide an excess of power which if needed from time to time can be used on-site, but in normal operation this excess power can be sold back to the grid.

The feedstock 101 is provided by regional growers 105 which as mentioned above is preferably on site with the processing facility that produces the protein meal 103 and fuel 104 from the feedstock 101 but not necessarily so. That processing facility will generate wastewater 106 which is treated to serve as a fertilizer 107 for growing the next generation of feedstock at the regional growers 105.

According to an example embodiment, Organic Protein Meal is produced by extracting the starch thus concentrating the protein from the Aquatic Plant Spirodela Polyrhiza and other aquatic plants. Starch extraction is accomplished using natural enzymes such as Alpha Amylase and Glucoamylase. The Aquatic plant Spirodela Polyrhiza is grown in a certified-organic and nitrogen rich water supply. This water supply is furnished as a byproduct of the starch extraction process. The starch is fermented to produce ethanol and yeast, the yeast. production giving further value to the final protein concentration. This final concentrated protein meal is organic and can serve as an alternative to other market livestock and fish feeds such as soybean meal that is both more economical and more environmentally friendly to produce.

The concentrated protein meal produced according to these teachings can be done by recycling the nitrogen rich waste water 106 that results from the ethanol production after the solids are removed and turned into the protein meal. Duckweed can be aquatically grown year round in the proper climate and with this nitrogen rich water recycled back to the duckweed grow ponds it can be harvested multiple times per week. The volume production of duckweed meal per acre according to these teachings can therefore far exceed that of soybean meal from grown soybean plants, and when grown year round can address winter shortages that routinely arise in the organic soybean market.

Spirodela Polyrhiza (giant duckweed) is currently used in sewage treatment and its grow rates are well documented. However, to the inventor's knowledge growing this aquatic plant as an organic protein source has never been considered because no reasonably available and economically viable fertilizer source exists to do so. Currently only the protein, nitrogen sources of soybean meal, fish meal and other minor sources could be used to fertilize an organic duckweed growing system but these are too costly. When using the starch extraction process as described below where the feedstock 101 is spirodela polyrhiza/duckweed, ethanol or some other form of fuel 102 is stripped in the production process leaving a wastewater stillage 106 which is fermented. The solids are separated from that wastewater for protein meal recovery and the remaining thin stillage is a nitrogen rich process water that is recycled to the duckweed growing area thus proving an organic sources of fertilizer for growing the next generation of duckweed.

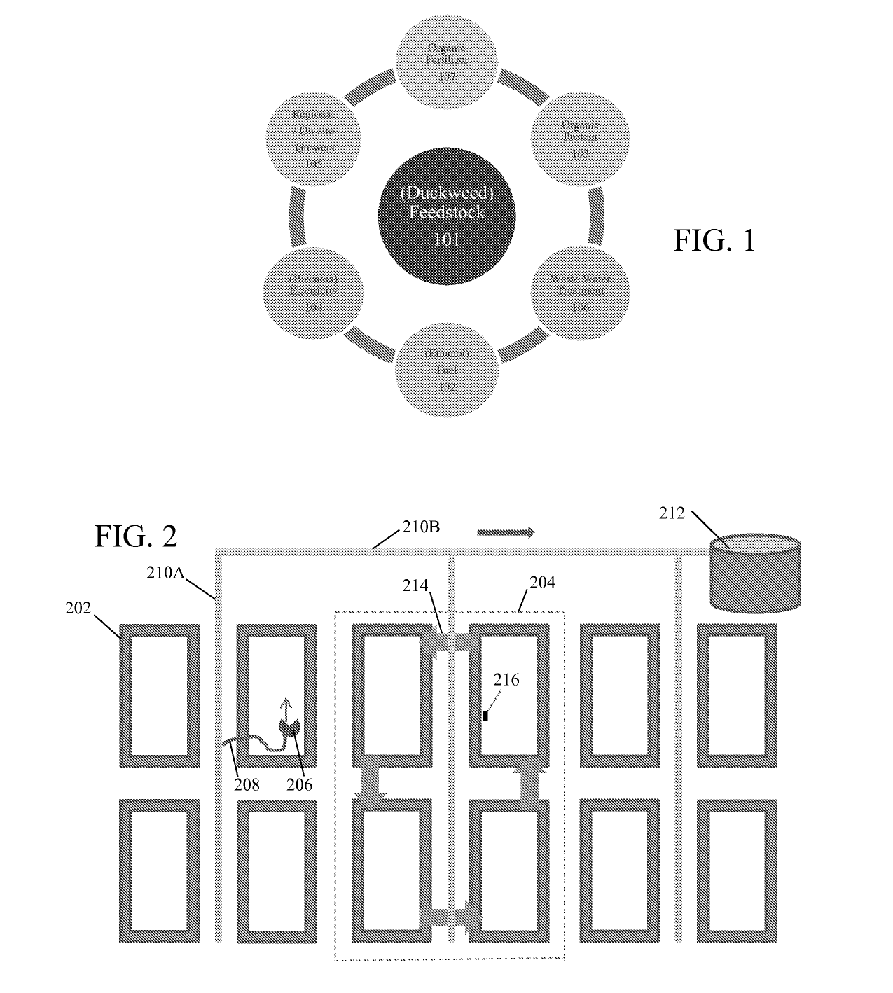

FIG. 3 shows a more detailed flow of this process for producing ethanol, protein meal and nitrogen rich fertilizer water, but first is described with reference to FIG. 2 how the lemna/duckweed or other aquatic feedstock 101 is grown in open ponds and collected. Across the southern United States duckweed is used in open ponds for sewage treatment since sewage water typically has high nitrogen levels. Being an aquatic plant, when grown as a feedstock 101 for the production for ethanol and protein meal the duckweed can be harvested from the grow ponds 202 via a remotely operated or even a fully automated floating surface skimmer 206 in which a submersible pump is mounted on a floating platform so as to skim the small duckweed plants from the surface of the grow pond 202. The grow ponds 202 may be constructed above ground for example with concrete sidewalls and floor, or partially below ground or at ground level with similar construction material or with some other material for which the sidewalls and floor are sealed by a distinct water-impermeable barrier such as a painted/sprayed/troweled coating, rubber/plastic sheeting or clay.

The skimmer 206 is moving in the direction indicated by the arrow and the submersible pump may be powered by a portable power source such as a galvanic battery disposed on-board the floating platform, or it may be connected by wire to an AC power supply. The skimmer 206 may be driven by an onboard electric motor such as an outboard trolling motor commonly used for fishing, and is preferably steered remotely via wireless control signals that in some embodiments are manually entered or in other embodiments are automatically generated based on GPS positioning of the skimmer or based on a pre-programmed harvesting pattern that the skimmer 206 follows about the grow pond 202. In a further embodiment the motor steers the skimmer randomly about the pond 202 and steers it away from sidewalls when it makes contact or comes within a prescribed minimum distance from them. Preferably the trolling motor is controlled to both forward motion at which the duckweed is most efficiently harvested, and reverse motion to back the skimmer 206 out of corners when necessary. Such a trolling motor can draw power from the same galvanic or AC source as the pump.

Flexible tubing 208 remains attached to the submersible pump while the skimmer 206 moves about the grow pond 202 and this flexible tubing 208 is attached at its opposed end to collection pipes 210A which are preferable raised in elevation so that the harvested duckweed can flow via gravity towards the collection bin 212. The duckweed is harvested by the skimmer via suction from the submersible pump and passes through the flexible tubing 208 via vacuum pressure to overcome gravity in getting to the nearest raised collection pipe 208A. While FIG. 2 shows only one harvesting skimmer 206, there may be a separate skimmer 206 in each duckweed grow pond 202 to avoid the manual labor of moving a skimmer between different grow ponds 202.

Alternative to the floating skimmer 208 that moves across the surface of the grow pond 202, there may be a fixed harvest point in each grow pond where the intake of a submersible pump is located. In this case the submersible pump may be in a fixed location and surface sweeper arms move the floating duckweed towards that fixed-location intake for removal from the grow pond 202.

In either case, while the harvested duckweed is taken up into the flexible tube 208 and/or while moving through the collection pipe 210A, the harvested duckweed may be partially dewatered via drain holes or screening that allows excess water to drain back into the grow pond 202 with only limited loss of the actual duckweed plants themselves.

Preferably the grow ponds 202 are in hydraulic communication via interconnecting pipes or controlled overflow gates indicated by the wide arrows 214. This does two things, it keeps a continuous though preferably slow circulation of water within each grow pond 202 to ensure the nitrogen fertilizer water from the ethanol production process that is recycled back into the grow ponds 202 is dispersed relatively evenly throughout each pond, and by interconnecting several grow ponds 202 a fewer number of sensors 216 are needed in the growing system to continuously or periodically monitor the levels of nitrogen and other nutrients on which the growing duckweed feeds. Feedback from these sensors 216 can be used to more precisely regulate optimum nutrient levels for optimizing growth of the duckweed, and the nutrient levels can be adjusted by how much nitrogen rich water is recycled back or how much the thin stillage is diluted before being recycled back to the grow ponds 202.

For robustness against disease or other threats to the growing duckweed it is preferable that for a given growing system the entirety of the duckweed grow ponds 202 are not all in hydraulic communication but rather the whole consists of several mutually exclusive subsets 204 of duckweed grow ponds 202 where ponds 202 within a given subset 204 are all in hydraulic communication with one another but the different subsets are hydraulically isolated from one another. In FIG. 2 one non-limiting example of such a subset 204 is identified by the dashed lines that enclose four grow ponds 202. For harvesting the collection pipes 210A associated with one given subset may be interconnected via crossover piping 210B so that duckweed harvested from multiple subsets 204 are collected at a single collection facility/bin 212.

In some embodiments the duckweed or other feedstock is not grown on site but is purchased from third party growers or distributors and imported to the ethanol/protein meal production facility represented by FIG. 3. But due to the nitrogen-rich thin stillage produced by the ethanol and protein meal production process described below it is preferable the feedstock is grown on site or nearby since this thin stillage can be recycled to grow the next generation of feedstock.

If the thin stillage is to be shipped off-site for growing feedstock at a location remote from the ethanol/protein meal production facility, preferably there is a sensor 216 (as detailed above for the grow pond 202) within the holding tank or other retention facility that holds the thin stillage to monitor levels of nitrite and/or nitrate as well as other nutrient levels. These readings can be used as feedback to adjust the process for making the ethanol and protein meal as will be detailed below.

Production of ethanol and protein meal from the harvested duckweed is now described in detail with reference to FIG. 3. The collected lemna/duckweed or other (preferably starch-based) feedstock moves, in order, through the following main processes:
milling 302;
hydrolysis 304/saccharification 306;
fermentation 308 which produces carbon dioxide 310 as an output from the system;
distillation 312 and dehydration 314 from which ethanol 316 is produced as an output from the system; and
separation/centrifuging 318 which yields
   a concentrated protein meal 322 from the solids which are dried 320 and output from the system; and
   a nitrogen-rich thin stillage 324 which is recycled back to the grow ponds described with reference to FIG. 2 or to other feedstock growing areas.

The feedstock is dewatered first in the milling process 302 to remove excess water; this is not a drying process but can be a simple removal of excess water by draining via gravity or this dewatering process may spin the harvested feedstock in a centrifuge for a more thorough as well as time-efficient dewatering. In the inventors' experiments the wet duckweed entering the process line of FIG. 3 is about 8% solids, so reducing the water content early in the FIG. 3 process makes all the remaining stages more efficient. This dewatering better prepares the feedstock for the salient part of the milling process 302 which uses two methods to mill the feedstock, mechanical grinding such as via a colloid mill that shears the solids or other such mechanical grinding mechanism that reduces the average particle size of the feedstock solids, followed by sonication such as via an ultrasonic device that essentially explodes the cells that form the solids. In general the milling 302 can be considered a two-stage process where the first stage (grinding) reduces particle size generally without breaking the cellular structure of the feedstock, and the second stage breaks the cell membranes/cell walls of the feedstock molecules.

In general, within a processing facility according to these teachings as described with reference to FIG. 2 the functions of the milling process 302 may be performed by milling equipment arranged to reduce particle size of a feedstock and rupture cell walls of the feedstock. Colloid grinders and sonicators are examples of such milling equipment.

The hydrolysis 304 and saccharification 306 stages are actually done together but explained separately for clarity. The hydrolysis stage 304 is enzymatic hydrolysis and represents the addition of enzymes to the ground feedstock/lemna. In the specific process used by the inventor the two enzymes are those mentioned above, namely alpha amylase and glucoamylase. The enzymatic hydrolysis 304 and saccharification 306 stages together convert the starch in the feedstock, which is more fully exposed by having the cell walls ruptured, into sugar. In conventional bioethanol processes this is done in a sugaring tank. There are other enzymes known to be suitable for this purpose, known both in the arts related to bio-ethanol production and related to craft brewing of beer.

Raising the temperature of the cell-ruptured feedstock solids with the enzymes added thereto is represented by the saccharification stage 306. In one embodiment this heating is done in-line in the process piping that moves the cell-exploded duckweed solids from the sonicator of the milling stage 302 to the sugaring tank where the hydrolysis 304 and saccharification 306 stages occur. This hot duckweed with the enzymes then sits in the sugaring tank for approximately one hour (about or approximately as used herein is +/−10%) at a temperature of 230° F. (110° C.), but the time can vary greatly depending on how high is the temperature in the sugaring tank and how complete is the conversion of starch in the solids that are input to the tank to sugar in the slurry that is output from it. The temperature and time profile for retention in the sugaring tank can be adjusted to tailor the output of protein at 322 to a concentration of 40-61% since the protein concentration correlates directly to how thorough is that conversion of starch to sugar.

In general, within a processing facility according to these teachings the functions of the hydrolysis and saccharification processes 304 and 306 may be performed by sugaring process equipment arranged to cook outputs from the milling equipment so as to convert starch in those outputs to sugar. A sugaring tank is an example of such sugaring process equipment.

Between the sugaring tank (hydrolysis 304 and saccharification 306) and the fermentation tank 308 there is preferably a heat exchanger to reduce the temperature to approximately 80° F. (27° C.). This cooling can be done by simply waiting rather than an industrial heat exchanger process, the key purpose is to ensure the slurry to which yeast is added at the fermentation stage 308 is cool enough so that the living yeast fungi itself survives being added to the slurry. In general this function may be performed by a heat exchanger, disposed between the sugaring process equipment at 304/306 and the fermentation equipment at 308, that is adapted to reduce temperature of the output of the sugaring process equipment from greater than 200° F. to less than 100° F.

In the fermentation stage 308 preferably the yeast and the slurry combination lay in the fermentation tank for about 24 hours during which the sugar is converted to ethanol. As the yeast grows in the fermentation process 308 it produces carbon dioxide 310 which is output from the system. The amount of yeast and the time to ferment in this stage 308 is a function of the nitrogen cycle which is monitored as described below; in the fermenting tank the fermentation process changes the form of nitrogen from elemental nitrogen to nitrite and/or nitrate which is a form that plants can uptake. The amount of yeast and the time to ferment are adjusted so as to optimize the nitrate/nitrite content in the autolyzed yeast water that is recycled back to the grow ponds 202 for growing duckweed or other feedstock 101.

In general, within a processing facility according to these teachings the functions of the fermentation process 308 may be performed by fermentation equipment arranged to ferment outputs of the sugaring process equipment so as to convert sugar in said outputs to protein while converting elemental nitrogen to nitrate and/or nitrite. Such nitrogen conversion is detailed further below, and a fermentation tank is an example of such fermentation equipment.

The output of the fermentation process 308 back into the process line of FIG. 3 is a stillage which has both solids and liquids. Preferably the liquids are skimmed or centrifuged from the solids so that only the liquids are run though the actual distillation columns at the distillation stage 312. In this case the liquids output from the fermentation stage 308 can be referred to as a beer. In the inventor's experience with duckweed as a feedstock this beer is about 8-10% ethanol. This beer is processed in a distillation column 312 and further dehydrated 314 such as via a molecular sieve or vapor separation to strip water from the ethanol via their different boiling points. The output from this distillation 312/dehydration 314 process is ethanol 316 which can be sold commercially to a refiner for mixing with petroleum products for various retail uses, or it may be used on-site as a fuel source for an on-site electrical power generator as mentioned above.

In general, within a processing facility according to these teachings the functions of the distillation and dehydration processes 312 and 314 may be performed by distillation equipment arranged to strip an alcohol based fuel from outputs of the fermentation equipment. A distillation column with optionally a molecular sieve is an example of such distillation equipment.

The solids from the fermentation process 308, and any liquids other than the ethanol from the distillation 312/dehydration 314 processes, is run through a separation/centrifuge process 318 to more thoroughly separate the liquids from the solids. This is a thorough centrifuging since the resulting solids are then dried 320 (preferably freeze-dried) to result in the concentrated protein meal 322 that is output from the system. The liquids resulting from the centrifuge process 318 is a thin stillage that is rich in nitrogen and can be recycled 326 back to the duckweed grow ponds after proper dilution. Any soluble solids 324 from the centrifuge 318 may be put back as inputs to the process line of FIG. 3 as shown.

In general, within a processing facility according to these teachings the functions of the separation/centrifuging process 318 may be performed by separation equipment arranged to separate solids and liquids from outputs of the distillation equipment (such outputs other than the stripped fuel 316), and from outputs of the fermentation equipment. A centrifuge is an example of such distillation equipment. Further, the dryer functions 320 may be performed in such a processing facility by drying equipment arranged to dry solids output from the separation equipment, and a freeze dryer is an example of such drying equipment.

Research into utilizing duckweed for primarily ethanol production, for example in place of maize-based feedstocks, have used duckweed extracted from pilot-scale water treatment processes such as lagoons of diluted pig effluent in North Carolina. Duckweed grown according to these teachings can be 100% organic, meaning the resulting protein meal can also be certified organic. In those experiments the duckweed starch content was increased to about 65% by retaining it in a well water for 10 days, and a 95% starch conversion rate was achieved. But no research has been found whereby the solids from this duckweed-to-ethanol process were used as an end product in and of itself, and particularly none has been found where it was made into a high concentration protein meal.

A substantial advantage realized with embodiments of these teachings is that the overall system recycles the autolyzed yeast water containing ruptured yeast nitrogen produced from the fermentation process 308. This yeast water is recycled back to the aquatic plant grow ponds 202 and can provide 100% of the nitrogen needed to grow the next batch of lemna/duckweed, making the process 100% sustainable and self-supporting.

Protein meal produced by this process can be a substitute for soybean meal, and one environmental advantage in doing so is elimination of fertilizers applied to the fields in which soybeans are grown, and the attendant runoff of excess amounts of that fertilizer into waterways. Another advantage is the elimination of the need for pesticides which might be used when growing such soybeans. Even where the need for pesticides is diminished through the use of genetically modified (GMO) soybeans, there is resistance to GMO food products and there does not appear to be any need to genetically modify at least duckweed when grown for processing according to these teachings. Unlike soybean meal from GMO soybean crops, the protein meal produced according to these teachings can be certified as organic.

Sugar based additives can also be added to the ground feedstock prior to the hydrolysis 304/saccharification 306 process to increase the sugar content of the product input to the fermentation tank 308. Sugar beet molasses can be used for this purpose, and the betaine that is naturally present in the sugar beets from which the molasses is made provides the added benefit of dramatically increasing the methionine content in the end result protein meal. Methionine is a sulfur-containing proteinogenic amino acid. For both human and livestock consumption the amino acid profile of any bulk protein source is important; for example 11 amino acids are considered essential for humans because these 11 cannot be synthesized from intermediary human metabolism and must be consumed. Methionine is one of the 11 human-essential amino acids. The needed amounts of the various amino acids differ from species to species and so the ideal or desired amino acid profile of protein meal for different species will also differ accordingly, where the amino acid profile specifies both the specific amino acids present in the protein meal as well as their relative concentrations or amounts.

From this discovery that adding betaine to the production process can greatly increase methionine in the end product protein meal follows the more general observation that further additives can be introduced prior to the fermentation stage 308 to achieve a desired amino acid profile in the end product protein meal. For example, assume the essential amino acids for chickens is represented by set A and that for dairy cattle are represented by set B. When producing protein meal for chickens a first group of additives is introduced into the sugaring tank 304/306 or elsewhere prior to the fermentation process 308 so that the amino acid profile present within the resulting protein meal has the appropriate levels of amino acid set A. Similarly when producing protein meal for dairy cattle a second group of additives is similarly introduced so that the amino acid profile present within the resulting protein meal has the appropriate levels of amino acid set B. These different amino acid profiles in the protein meal can be obtained from different batches of the same feedstock running through the same process line so the changeover from making meal for chickens to making meal for dairy cattle is very simple and efficient. Conventionally, obtaining the proper amino acid profile for a given species' protein meal is done at the feed mill which takes meal from different sources and mixes them in specified amounts to obtain the desired amino acid profile. These teachings enable one to obtain the desired amino acid profile by the addition of specific chemical reagents in specific amounts during the production itself of the protein meal from a feedstock. In this case the different amino acids or amounts in the protein meal produced for two different species are the products of chemical reactions consuming the added reagents, where the chemical reaction itself occurs in the process line (e.g., in the fermentation tank 308).

Above with reference to FIG. 2 it was detailed that in the grow ponds 202 there may be sensors 216 to continuously or periodically monitor nutrient levels in the water, specifically but not limited to nitrogen levels. In general plants such as duckweed cannot absorb elemental (meat or N₂) nitrogen. Nitrogen fertilizer for plants should be in the form of nitrite (NO₂) or nitrate (NO₃⁻). In order to get nitrogen in this form for recycling back to the grow ponds 202 the level of meat nitrogen in the fermenting tank 308 or prior to it is tested. This determines the amount of yeast to add to the fermenting tank 308 in order to get the proper conversion during fermentation from meat nitrogen to nitrite and/or nitrate since it is in the fermenting tank 308 that this conversion of N₂ to NO₂⁻ and/or nitrate NO₃⁻ takes place. Because the growth cycle of duckweed is so nitrogen intensive, monitoring this nitrogen cycle at various locations in the overall system (grow ponds 202 or other such liquid retention equipment), sugaring tank 304/306 or fermentation tank 308) is key to ensure an efficient process that is both self-sustaining and highly productive due to rapid growth of the duckweed feedstock. It is well documented that duckweed can double in size within 24 hours given sufficient nutrients and sunlight.

Whether the duckweed is grown on site or off the liquids output from the separation equipment 318 will need to be stored (for example, in the grow ponds 202 if onsite or in some other holding tank if not) in some kind of liquid retention equipment represented in FIG. 3 by block 324. In a facility according to these teachings this liquid retention equipment is arranged to store liquids output from the separation equipment and also has one or more nitrogen sensors to measure levels of nitrate and/or nitrite in the stored liquid. The grow ponds 202 are an example of such liquid retention equipment.

As can be seen from above the total process is an interconnected system; the recycled process water provides a readily available and economical nitrogen source to grow the duckweed feedstock, preferably organically. Recycling the autolyzed yeast water creates a closed nitrogen cycle which is highly economical and efficient, and currently no economical nitrogen source is known for growing duckweed as a commercial crop. The process is optimized by addition to the grow ponds 202 of various minerals but these needs are relatively minimal, particularly compared to the duckweed nitrogen uptake requirements to sustain its growth cycle. In experiments the inventor has grown duckweed via this process to approximately twice its naturally occurring size at the growth rates that are well documented already (e.g., doubling every 24 hours). To the inventor's knowledge the commercial production of organic duckweed has not been attempted elsewhere; duckweed has been used to treat sewage which generally is very nitrogen-rich and ethanol has been experimentally produced from duckweed grown from sewage but the solids left over from that ethanol production would not appear to qualify as organic and the inventor is unaware of any productive use that has been found for such solids.

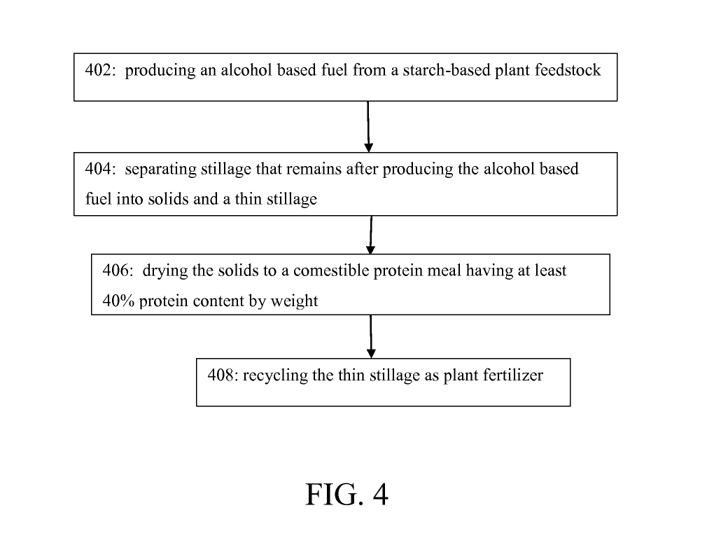

One particular aspect of a method according to these teachings is summarized at FIG. 4, which begins at block 402 with the general proposition of producing an alcohol based fuel such as but not limited to ethanol from a starch-based plant feedstock such as but not limited to duckweed. Block 404 summarizes what is described above as the separation or centrifuge stage, namely separating stillage that remains after producing the alcohol based fuel into solids and a thin stillage. At block 406 the solids are dried to a comestible protein meal having at least 40% protein content by weight, and in the examples above this protein content was between 40% and 61%. Finally at block 408 the thin stillage is recycled as plant fertilizer.

Above it was described that recycling the thin stillage includes using the thin stillage as a fertilizer for growing further amounts of the starch-based plant feedstock, but in fact this thin stillage is rich in nutrients for a wide variety of plants so recycling to grow the next batch/generation of feedstock is not a limiting aspect of these teachings; the thin stillage can be sold as an end product in its own right if some other starch-based plant feedstock is readily and abundantly available.

For the case in which the thin stillage is used in a closed system to fertilize more feedstock, then in some embodiments where producing the alcohol based fuel at step 402 includes a fermentation stage as detailed above, then an amount of yeast and/or an amount of fennentation time that defines that fermentation stage is controlled to optimize nutrients in the thin stillage for growing the further amounts of the starch-based plant feedstock. In the above non-limiting examples the nutrients that are optimized include at least nitrite and/or nitrate.

In a particular embodiment where producing the alcohol based fuel at step 402 includes a fermentation stage as detailed above, the method summarized at FIG. 4 further includes determining a desired amino acid profile for the comestible protein meal (for example, the desired profile may be for chickens, or for dairy cattle, or for humans); and adding at least one reagent prior to the fermentation stage so as to adjust an amino acid profile of the comestible protein meal closer to the desired amino acid profile due to addition of the at least one reagent. In this manner the amount of one or more amino acids that is output from the fermentation stage is tailored to match an amount of the same amino acid(s) in the desired amino acid profile; in this case since the fermentation tank processes in batches the ‘amount’ here refers to concentration. In the non-limiting examples above one such reagent is betaine which was added in the form of sugar beet molasses, and the amino acid profile of the comestible protein meal is adjusted to exhibit an increased amount of methionine, where methionine is produced in the fermentation process from the added betaine reagent.

What is claimed is:

1. A processing facility comprising:
milling equipment arranged to reduce particle size of a feedstock and rupture cell walls of the feedstock;
sugaring process equipment arranged to cook outputs from the milling equipment so as to convert starch in said outputs to sugar;
fermentation equipment arranged to ferment outputs of the sugaring process equipment so as to convert sugar in said outputs to protein while converting elemental nitrogen to nitrate and/or nitrite;
distillation equipment arranged to strip an alcohol based fuel from outputs of the fermentation equipment;
separation equipment arranged to separate solids and liquids from outputs of the distillation equipment other than the stripped fuel, and to separate solids and liquids from outputs of the fermentation equipment;
drying equipment arranged to dry solids output from the separation equipment; and
liquid retention equipment arranged to store liquids output from the separation equipment and having a nitrogen sensor to measure levels of nitrate and/or nitrite in the stored liquid.

2. The processing facility according to claim 1, further comprising a heat exchanger, disposed between the sugaring process equipment and the fermentation equipment, adapted to reduce temperature of the output of the sugaring process equipment from greater than 200° F. to less than 100° F.

3. The processing facility according to claim 1, wherein the drying equipment comprises freeze dryers.

4. The processing facility according to claim 1, wherein the liquid retention equipment comprises grow ponds configured to grow the feedstock and arranged to provide feedstock output from the grow ponds to the milling equipment.

5. The processing facility according to claim 4, wherein the feedstock is a starch-based aquatic plant.

6. The processing facility according to claim 4, further comprising floating skimmers in at least some of the grow ponds, each floating skimmer being remotely operated or fully automated and comprising an outboard electrically driven motor and a submersible pump configured to collect feedstock floating on a surface of the grow ponds.

7. A method for producing comestible protein meal, the method comprising:
producing an alcohol based fuel from a starch-based plant feedstock;
separating stillage that remains after producing the alcohabased fuel into solids and a thin stillage;
drying the solids to a comestible protein meal having at least 40% protein content by weight;
recycling the thin stillage as plant fertilizer.

8. The method according to claim 7, wherein recycling the thin stillage comprises using the thin stillage as a fertilizer for growing further amounts of the starch-based plant feedstock.

9. The method according to claim 8, wherein producing the alcohol based fuel from the starch-based plant feedstock comprises a fermentation stage, wherein an amount of yeast and/or an amount of fermentation time defining the fermentation stage is controlled to optimize nutrients in the thin stillage for growing the further amounts of the starch-based plant feedstock.

10. The method according to claim 9, wherein the nutrients that are optimized comprise at least nitrite and/or nitrate.

11. The method according to claim 7, wherein producing the alcohol based fuel from the starch-based plant feedstock comprises a fermentation stage, the method further comprising:
determining a desired amino acid profile for the comestible protein meal; and
adding at least one reagent prior to the fermentation stage so as to adjust an amino acid profile of the comestible protein meal closer to the desired amino acid profile due to addition of the at least one reagent.

12. The method according to claim 11, wherein the at least one reagent is betaine and the amino acid profile of the comestible protein meal is adjusted to exhibit an increased amount of methionine.

13. A comestible protein meal produced by the method according to claim 7.

14. A method for producing comestible protein meal, the method comprising:
determining a desired amino acid profile;
while producing an alcohol based fuel from a starch-based plant feedstock in a process that comprises a fermentation stage, adding at least one reagent prior to the fermentation stage so as to adjust an amount of at least one amino acid output from the fermentation stage to match an amount of the at least one amino acid in the desired amino acid profile;
separating stillage, containing the amount of the at least one amino acid, that remains after producing the alcohol based fuel into solids and a thin stillage; and
drying the solids to a comestible protein meal comprising the amount of the at least one amino acid.

15. The method according to claim 14, the method further comprising recycling the thin stillage as plant fertilizer.

16. The method according to claim 15, wherein recycling the thin stillage comprises using the thin stillage as a fertilizer for growing further amounts of the starch-based plant feedstock.

17. The method according to claim 16, wherein an amount of yeast and/or an amount of fermentation time defining the fermentation stage is controlled to optimize nutrients in the thin stillage for growing the further amounts of the starch-based plant feedstock.

18. The method according to claim 17, wherein the nutrients that are optimized comprise at least nitrite and/or nitrate.

19. The method according to claim 14, wherein the at least one reagent is betaine and the at least one amino acid is methionine, and wherein the amount of methionine output from the fermentation stage is increased due to the betaine being added prior to the fermentation stage.

20. A comestible protein meal produced by the method according to claim 14.
